(19) United States
(12) Patent Application Publication
(10) Pub. No.: US 2002/0019550 A1
(43) Pub. Date: Feb. 14, 2002

(54) Process for the preparation of alpha-sulfonated polyhydric alcohol esters

(76) Inventors: Salmiah Ahmad, Selangor Darul Ehsan (MY); Adam Ibrahim Ahmed Nur, Jersey City, NJ (US); Mohd Zaizi Desa, Selangor Darul Ehsan (MY); Dzulkefly Kuang, Selangor Darul Ehsan (MY)

Correspondence Address: Finnegan, Henderson, Farabow,, Garrett & Dunner, L.L.P., 1300 I Street, N.W., Washington, DC, 20005-3315, US

(21) Appl. No.: 09/892,573

(22) Filed: Jun. 28, 2001

(30) Foreign Application Priority Data
Jun. 28, 2000 (MY) PI 2000 2978

(51) Int. Cl. C 07C 3 9/17

(52) U.S. Cl. 554/98

(57) ABSTRACT

A process for the production of alpha-sulfonated polyhydric esters (ASPA) is disclosed using starting materials derived from palm oil or palm kernel oil. The process involves reacting saturated alkyl carboxylic acids or, alkyl esters with sulfonating agent, such as gaseous SO₃ followed by reaction with stoichiometric amount of polyhydric alcohols. The resultant product may be bleached with bleaching agent, such as H₂O₂ and neutralised with an aqueous base to produce water soluble alpha-sulfonated polyhydric alcohol ester salt (ASPA). The product is obtained with good color and yield. This process is more efficient as it requires only stoichiometric amounts of reagents, and can reduce the formation of undesirable by-product such as sodium sulfonated soaps.

### FIELD OF THE INVENTION

This invention relates to a process for preparing alpha-sulfonated polyhydric alcohol esters (ASPA).

It also relates to products prepared according to the process.

### BACKGROUND OF THE INVENTION

U.S. Pat. No. 5,319,117 discloses a process for the production of sulfonated fatty acid glycerol esters by the reaction of unsaturated fatty acid glycerol esters with gaseous sulfur trioxide followed by neutralisation with aqueous base and subsequent heating and then phase separation. However, as this process uses unsaturated fatty acid esters this does not lead to an alpha-substituted product. When an unsaturated fatty acid chain is used the sulfonation occurs across the double bond of the unsaturated fatty acid.

U.S. Pat. No. 4,671,900 discloses a process for preparing monoalkyl esters of alpha-sulfofatty acids. However, there is no suggestion in this patent of forming esters of alpha-sulfofatty acids with polyols.

Micich et al., JAOCS Vol 49 (1972) at the right-hand column of page 90, describes the sulfonation of stearic acid in a flask using dioxane and carbon tetrachloride as solvents. Sulfur trioxide in carbon tetrachloride was added to the stearic acid and the mixture was heated for 1 hour at 60° C. The sulfostearic acid (1 mole) that was formed in the carbon tetrachloride solution was then esterified with two moles of pentaerythritol. The resultant product was then neutralised with 5N NaOH to form the sulfonate sodium salt.

Bistline et al., JAOCS 46 (1969) at pages 540 to 550 describes three procedures that can be used to produce polyhydric alcohol esters of alpha-sulfo acids. In the first procedure a mixture of 2.5 mole of D-mannitol was added to 1 mole of alpha-sulfostearic acid and refluxed in benzene for 4 hours. The product was then neutralised with 18N NaOH. In the second procedure 1 mole of sucrose dissolved in dimethylformamide and pyridine was reacted with 1 mole alpha-sulfopalmitoyl chloride dissolved in carbon tetrachloride. 50% aqueous ethanol was added followed by neutralisation with 18N NaOH. The monoester yield was 36%. In the third procedure, a mixture of sucrose and alpha-sulfostearate was dried at 100° C. under a vacuum of 1 mm of Hg and then dissolved in dimethylformamide in the presence of sodium methoxide as the catalyst. The mixture was heated and stirred for 6 hours at 100° C. to form an ester product.

In a further method, alpha-sulfo acid was reacted with benzyl alcohol, cyclohexane or phenol in the presence of methylene chlorde, various solvents such as ethanol, carbon tetrachloride or toluene were used (Bistline et al JAOCS Vol 45 (1968) page 78).

In the above prior art procedures at least one of the following disadvantages are evident.

1) formation of considerable amount of disalt which degrades the performance of the product;

2) formation of diesters or polyesters;

3) formation of hydrogen chloride, which may cause difficulties and incur added costs for its disposal;

4) use of organic solvent, which increases the cost and requires the subsequent removal of the solvent;

5) poor colour development due to higher temperature used to evaporate and remove organic solvent;

6) use of harmful and flammable chemicals.

7) formation of monoalkyl esters.

### SUMMARY OF THE INVENTION

This invention provides in one form a process for the production of alpha-sulfonated polyhydric alcohol ester comprising the steps of:

A) reacting fatty acid or fatty acid methyl ester with gaseous sulfur trioxide to produce acidic alpha-sulfonated fatty acid or alpha sulfonated fatty methyl ester;

B) reacting glycerol with acidic alpha-sulfonated fatty acid or acidic alpha- sulfonated fatty methyl ester to produce acidic alpha-sulfonated polyhydric alcohol ester (ASPA);

C) optionally aging the acidic ASPA at elevated temperature;

D) optionally bleaching the acidic ASPA with hydrogen peroxide; and

E) neutralising it with aqueous base.

Preferably the fatty acid or the fatty methyl ester is of natural origin or synthetically produced and contains from 8 to 18 carbon atoms.

Preferably the fatty acid or the fatty methyl ester includes a saturated hydrocarbon chain that is derived from vegetable oil or animal fat, such as palm oil, palm kernel oil, coconut oil or tallow.

Preferably the fatty acid or fatty methyl ester has an iodine value less than 0.5.

Preferably the fatty acid or fatty methyl ester is derived from hydrogenated palm oil, palm kernel oil, coconut oil or tallow.

Preferably step A) is carried out in a falling film reactor.

Preferably in step B) the molar ratio of glycerol to the sulfonated products is from about 1 to 1.2.

Preferably in step B) the reaction of alpha-sulfonated fatty acid with glycerol is carried out at a temperature in the range of 40 to 90° C.

Preferably in step B) the reaction of alpha-sulfonated fatty methyl ester with glycerol is carried out at a temperature in the range of 50 to 90° C.

Preferably in step B) the reaction of alpha-sulfonated fatty methyl ester with glycerol is carried out at a reaction time of 30 to 90 minutes.

Preferably step C) is carried out from a period of 1 to 60 minutes.

Preferably step D) is carried out using hydrogen peroxide.

Preferably in step D) the amount of hydrogen peroxide is 3 to 4%.

Preferably in step E) is aqueous sodium hydroxide.

Preferably in step E) is carried at pH from 7 to 8.

The optional bleaching step D) may take place before the glycerol esterification step.

In an alternative form the invention provides a process for the production of alpha-sulfonated polyhydric alcohol ester comprising the steps of:

I sulfonating a saturated fatty acid or fatty methyl ester to form an alpha-sulfonated fatty acid or an alpha-sulfonated fatty acid methyl ester.

H esterifying the reaction product of stage I with a polyhydric alcohol having at least two hydroxyl groups and wherein the mole ratio of polyhydric alcohol to the reaction product of stage I is in the range 0.8: 1 to 1.4: 1 and more preferably 1.1:1 to 1.2:1.

III neutralising the acidic alpha-sulfonated fatty esters of the polyhydric alcohol with an aqueous base.

Preferably the process includes steps C and D as described above between stages II and III.

Preferably the polyhydric alcohol is selected from the group consisting of ethylene glycol, sorbitol, mannitol, sucrose, dextrose, glycerol, pentaerythritol, neopentyl glycol and trimethylolpropane.

Preferably the aqueous base is formed from an alkali metal, and more preferably sodium.

Preferably the fatty acid radical is derived from chains of C8 to C24 carbon atoms.

More preferably the fatty acid radical is derived from chains of C12 to C18 carbon atoms.

In a further form this invention provides a product prepared by a process as described above.

### DETAILED DESCRIPTION OF THE INVENTION

The following equations illustrate the reactions of the present invention with glycerol as an example.

Stage I—Sulfonation

a) R—(CH₂)ₙ₊₁COOCH₃+2SO₃→R—(CH₂)ₙCHSO₃HCOOCH₃+SO₃

b) R—(CH₂)ₙ₊₁COOH+SO₃→R—(CH₂)ₙCHSO₃HCOOH

Stage-2 Glycerolysis

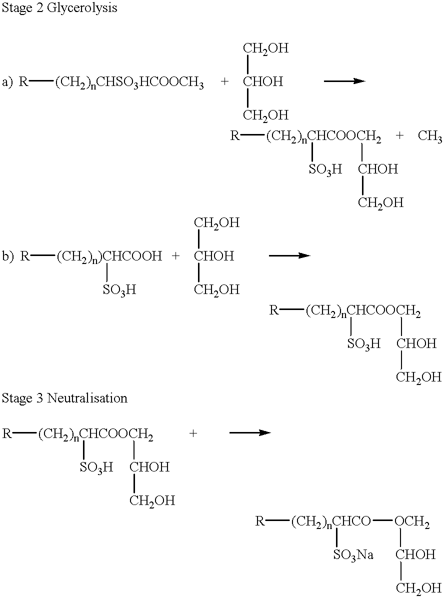

In stage 1 fatty acids or fatty methyl esters art used as starting materials for the preparation of ASPA. Straight chain and saturated monocarboxylic acids or their esters containing 6 to 18 carbon atoms are preferably used. Especially preferred are C12 (Lauric), C14 (Myristic), C16 (Palmitic), and C18 (Stearic). These acids can be derived from natural products such as palm oil, palm kernel oil, coconut oil or tallow. Methyl esters are obtained either by esterification of the corresponding fatty acids or by transesterification of oils or fats with methanol. The fatty acid natural products contain unsaturated fatty acids and it is preferred that the fatty acids or esters are hydrogenated to an iodine number less than 0.5. The fatty acids or methyl esters are sulfonated by known methods to prepare acidic alpha-sulfonated fatty acid or alpha-sulfonated fatty ethyl ester. The molar ratio of fatty acid or fatty methyl ester to sulfonating agent is preferably in the range of 0.8 to 1.4, and more preferably to 1 to 1.2. The sulfonation of the fatty acids or methyl esters may be achieved using a gaseous mixture of sulfur trioxide in dry nitrogen at a temperature in the range of 80 to 90° C., preferably 80° C. Generally, the higher the temperature, the greater will be the degree of sulfonation However, the colour may be unsatisfactory if temperature is too high. The absorption of 2 moles of SO₃ by fatty methyl esters to form an adduct is rapid. The adduct, upon digestion for 15 to 40 minutes, preferably 30 minutes, at a temperature of 60 to 70° C., releases a mole of SO₃ to form the acidic alpha-sulfonated methyl ester (Kapur et al., JAOCS Vol 55, 1978 page 550). If the adduct is not digested, prior to polyolysis, hydrolysis occurs during neutralisation to form sodium sulfonated soap (disalt) which degrades the performance of the product. Due to the excess of SO₃ used during the sulfonation process, some of the adduct still remain even after digestion and can form disalt during neutralisation. The formation of this disalt can be further minimised by reesterification with an alcohol or polyol.

The absorption of SO₃ by fatty acid occurs in two steps: (Kapur et al., JAOCS Vol 55, 1978 pages 549 to 557) first, formation of mixed anhydride and second rearrangement of mixed anhydride at a higher temperature to form alpha-sulfonated fatty acid. The product obtained is viscous and dark in colour.

In the second stage glycerol or other polyhydric alcohols are added to the alpha-sulfonated fatty acid at temperatures Generally in the range of 50 to 90° C., more preferably 40 to 60° C. Alternatively it is added to the alpha-sulfonated fatty methyl ester at a temperature in the range of 50 to 90° C., more preferably 50 to 70° C. The proportions of the sulfonated product and glycerol are preferably in the molar range of 0.8 to 1.2 and more preferably 1 to 1.1. The reaction mixture was then stirred (100 rpm) over a period of 30 to 90 minutes preferably 60 minutes. The water or methanol, which is produced during the reaction, may be removed by venting using vacuum of 50-80 mbar. The product obtained is designated acidic ASPA. One of the advantages of the process of the present invention is regardless of whether fatty acid or fatty methyl ester is used, the final product is ASPA. If fatty acid is used the process completely avoids the formation or release of methanol, which is a flammable compound.

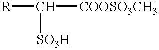

The level of disalt formed is generally less than 3-4% w/w.

Typically, the reaction product is a dark coloured ASPA acid. This product may be aged for 20 to 30 minutes at temperatures of 50 to 60° C. and then, bleached to a light yellow colour with 30% hydrogen peroxide. Aqueous hydrogen peroxide is usually added incrementally to the sulfonated product with constant stirring. The amount of H₂O₂ may by 3 to 4%, preferably 4% (based on the weight of sulfonated product) at a temperature of 55 to 60° C., and bleaching time is in the range of 10 to 40 minutes, preferably 30 minutes. The bleaching is temperature dependent. However, conditions that permit product hydrolysis should be avoided. After bleaching with hydrogen peroxide, the resultant product can be dissolved in water or a lower alkyl alcohol such as methanol, under constant stirring. The methanol reduces the viscosity of the reaction product and facilitates smooth agitation. Methanol can be recovered if desired by vaporisation and condensation.

In stage 3, neutralisation is carried out using a 30% solution of sodium hydroxide (based on weight) with vigorous agitation. The pH is maintained in the range of 7 to 8, preferably 7. The brown colour of the product generally disappears to produce a yellow gel alpha-sulfonated polyhydric alcohol ester (ASPA) salt.

The washing active substance (WAS) was found to be 70%. It was found that ASPA salt prepared according to the present invention has a superior performance in terms of surface tension.

ASPAs are anionic surfactants, which were found to be effective detergents and lime soap dispersing agents. These esters are considerably more water soluble than the corresponding sulfonated fatty esters, and can tolerate polyester impurities without decreasing water solubility or surfactant properties (Bistline et.al, JAOCS Vol 46, 1969, page 549). The product obtained according to the invention is valuable as surfactants because they can reduce the sodium-sulfonated soaps.

The invention will be further described by reference to the following examples which illustrate preferred embodiments.

### EXAMPLE 1

Production of ASPA from fatty methyl ester

The fatty methyl ester C12 (256.6 g, 1.2 moles) was pumped (10 g/min) to the tope of a 600 g/hr pilot plant sulphonation reactor. Oleum (571 g) was pumped (22.4 gimin) into the evaporiser maintained at 150° C. The liberated SO₃ was 20% (based on oleum weight) and the latter was diluted with compressed dry N₂ (125 L/hr). The streams of dry N₂ also help to push the SO₃ to the top of the reactor. The organic feed (fatty methyl ester) was heated to 50° C. and diltted gaseous SO₃ concurrently enters the reactor (reaction zone) where the two streams meet and sulfonation is initiated at a temperature of 90° C. The reactor effluent was collected into the receiver. The product was aged for 30 minutes to complete the reaction. The dark coloured product obtained is referred to as acidic alpha-sulfonated methyl ester (acidic SME). Acidic SME was placed in a IKA reactor. Glycerol (33 g, 0.35 moles) was added drop-wise to the acidic SME (88.38 g, 0.3 moles). The reaction was carried out at 70° C. for 50 neutes under constant stirring (100 rpm). A vacuum (50-80 mbar) take-off line was connected to reaction system to remove methanol from the reaction environment. The product obtained, known as acidic ASPA or ASPA acid was dark in colour. Acidic ASPA was subjected to aging and then bleached with 30% hydrogen peroxide. The amount of hydrogen peroxide used was 4% (based on weight of sulfonated product) and bleaching time was 30 minutes at a temperature of 60° C. The bleached product was neutralised with 30% solution sodium hydroxide at a pH of 7 to 8. The methanol was removed by rotavapor. The final product obtained was a mixture of alpha-sulfonated polyhydric alcohol ester (ASPA) and alpha-sulfonated fatty methyl ester (minor amounts) in the form of yellowish colour gel.

### EXAMPLE 2

Production of ASPA from fatty acid

The sulfonation of fatty acid was carried out in the same manner as the fatty methyl ester from Example 1. However, the temperature of the organic column was 10 to 15° C. higher to liquefy the fatty acid, and the reaction column was maintained at 80° C. The product obtained is acidic alpha-sulfonated fatty acid and it was esterified with glycerol using the IKA reactor. Glycerol (33 g, 0.35 moles) was added to alpha-sulfonated fatty acid (88.38 g, 0.3 moles), at a reaction temperature of 40° C. under constant stirring (100 rpm) for 60 minutes, A vacuum (50-80 mbar) take-off line was connected to the reaction system to remove water from the reaction environment. The esterification process gave dark coloured product. The sulfonated product was subjected to aging, then bleached with 30% hydrogen peroxide. The amount of hydrogen peroxide used was 4% (based on weight of sulfonated product) and the bleaching time was 30 minutes at temperature of 60° C.). The bleached product was then neutralised with 30% solution sodium hydroxide at pH of 7 to 8. The final product obtained was alpha-sulfonated polyhydric alcohol ester (ASPA) in the form of yellowish coloured gel.

The claims defining the invention are as follows:

1. A process for the production of alpha-sulfonated polyhydric alcohol ester comprising the steps of:
A) reacting fatty acid or fatty methyl ester with gaseous sulfur trioxide to produce acidic alpha-sulfonated fatty acid or alpha sulfonated fatty methyl ester;
B) reacting glycerol with acidic alpha-sulfonated fatty acid or acidic alpha- sulfonated fatty methyl ester to produce acidic alpha-sulfonated polyhydric alcohol ester (ASPA);
C) optionally aging the acidic ASPA at elevated temperature;
D) optionally bleaching the acidic ASPA with hydrogen peroxide; and
E) neutralising the acidic ASPA with aqueous base.

2. The process of claim 1 wherein the fatty acid or fatty methyl ester is of natural origin or synthetically produced and contains 8 to 18 carbon atoms.

3. The process of claim 2 wherein the fatty acid or the fatty methyl ester includes a saturated chain that is derived from vegetable oil or animal fat, such as palm oil, palm kernel oil, coconut oil or tallow.

4. The process of claim 2 wherein the fatty acid or fatty methyl ester has an iodine value less than 0.5.

5. The process of claim 4 wherein the fatty acid or fatty methyl ester is derived from hydrogenated palm oil, palm kernel oil, coconut oil or tallow or hydrogenated fatty acid or methyl ester.

6. The process of claim 1 wherein step A) is carried out in falling film reactor.

7. The process of claim 1 wherein step B) the molar ratio of glycerol to the sulfonated products is from about 1 to 1.2.

8. The process of claim 1 wherein step B) the reaction of alpha-sulfonated fatty acid with glycerol is carried out at a temperature in the range of 40 to 90° C.

9. The process of claim 1 wherein step B) the reaction of alpha-sulfonated fatty methyl ester with glycerol is carried out at a temperature in the range of 50 to 90° C.

10. The process of claim 1 wherein step B) the reaction of alpha-sulfonated fatty methyl ester with glycerol is carried out at a reaction time of 30 to 90 minutes.

11. The process of claim 1 wherein C) is carried out from a period of 1 to 60 minutes.

12. The process of claim 1 wherein step D) is carried oft using hydrogen peroxide.

13. The process of claim 1 wherein step D) the amount of hydrogen peroxide is 3 to 4%.

14. The process of claim 1 wherein step E) is aqueous sodium hydroxide.

15. The process of claim 1 wherein step E) is carried at pH from 7 to 8.

16. A process for the production of alpha-sulfonated polyhydric alcohol ester comprising the steps of:
I sulfonating a saturated fatty acid or fatty methyl ester to form an alpha-sulfonated fatty acid or an alpha-sulfonated fatty methyl ester.
II esterifying the reaction product of state I with a polyhydric alcohol having at least two hydroxyl groups and wherein the mole ratio of polyhydric alcohol to the reaction product of stage I is in the range 0.8:1 to 1.4:1 and more preferably 1.1:1 to 12:1.
III neutralising the acidic alpha-sulfonated fatty esters of the polyhydric alcohol with an aqueous base.

17. A product prepared according to a process as defined in any one of claims 1 to 16.
